(19) United States
(12) Patent Application Publication
(10) Pub. No.: US 2013/0163723 A1
(43) Pub. Date: Jun. 27, 2013

(54) Method for Correctively Adjusting a Beam for Irradiating a Moving Target Volume

(71) Applicant: Martin Tacke, Erlangen (DE)

(72) Inventor: Martin Tacke, Erlangen (DE)

(21) Appl. No.: 13/725,238

(22) Filed: Dec. 21, 2012

(30) Foreign Application Priority Data
Dec. 23, 2011 (DE) DE 102011089748.8

(51) Int. Cl. A61N 5/10

(52) U.S. Cl. CPC A61N 5/1045
USPC 378/65; 378/151

(57) ABSTRACT

A radiotherapy device includes a therapeutic radiation source for providing a beam for irradiating a moving target volume and a beam correcting apparatus for directing the beam onto the target volume. The beam correcting apparatus includes a collimator having a collimator aperture for delimiting the beam, a positioning apparatus for positioning the target volume relative to the beam, and a controller for selecting a position of the collimator aperture relative to the beam and for selecting a position of the positioning apparatus such that when the beam is correctively adjusted, the movement of the target volume in a first movement direction is compensated for by displacing the collimator aperture, and the movement of the target volume in a second movement direction is compensated for by repositioning the target volume with the positioning apparatus.

This application claims the benefit of DE 10 2011 089 748.8, filed on Dec. 23, 2011, which is hereby incorporated by reference.

### BACKGROUND

The present embodiments relate to a method for correctively adjusting a beam for irradiating a moving target volume.

Radiation therapy is an established method of treating malignant tumors, where a high-energy treatment beam (e.g., high-energy x-ray radiation) is directed onto tissue that is to be irradiated (e.g., a tumor).

Radiation therapy for treating moving target volumes (e.g., a lung tumor) represents a challenge because the anatomy requiring to be irradiated moves. The aim is to apply the therapeutic radiation in as targeted a manner as possible to the tumor while sparing surrounding tissue as effectively as possible. The movement may reduce the accuracy of the irradiation because the target volume moves out of the focus of the treatment beam. This may result in an underdosage with respect to the radiation dose applied in the target volume, while surrounding healthy tissue is exposed to an excessively high dose.

One possibility of counteracting this uncertainty is to use a greater safety margin, though this may lead to healthy tissue encompassed by the safety margin being exposed to a stronger dose.

One method used in the irradiation of moving tumors is the method known as tracking. With the tracking method, the movement cycle of the target volume is monitored, and the beam is adjusted so that the beam continuously follows the movement of the target volume (e.g., the beam is corrected to track the moving target volume). The irradiation field is therefore adjusted automatically according to the movement of the target volume.

With the gating methods, the dose is applied only at specific instants in time (e.g., only when the target volume is located at predefined, planned positions).

### SUMMARY AND DESCRIPTION

The present embodiments may obviate one or more of the drawbacks or limitations in the related art. For example, a radiotherapy device that enables the beam to be corrected precisely and with little effort so as to track the movement of the target volume is provided.

The radiotherapy device includes a therapeutic radiation source for providing a beam for irradiating a moving target volume and a beam correcting apparatus for directing the beam onto the target volume. The beam correcting apparatus includes a collimator having a collimator aperture for delimiting the beam, and a positioning apparatus for positioning the target volume relative to the beam. The beam correcting apparatus also includes a control apparatus (e.g., a controller or a processor) for selecting a position of the collimator aperture relative to the beam and for selecting a position of the positioning apparatus such that when the beam is correctively adjusted, the movement of the target volume in a first movement direction is compensated for by displacing the collimator aperture, and the movement of the target volume in a second movement direction is compensated for by repositioning the target volume with the positioning apparatus.

In order to compensate for the movement of the target volume more accurately, two mutually independent subsystems are used simultaneously: the patient positioning apparatus and the collimator. The patient positioning apparatus and the collimator are used to correctively adjust the treatment beam in order to track the movement of the target volume. Each subsystem compensates for the movement of the target volume in a different direction.

In order to implement the combined tracking method, an integrated control system that actuates the collimator leaves and the positioning apparatus for the patient is used. The movement information supplied to the control system in relation to the movement of the target volume is analyzed by the control system in real time. The movement axes of the radiotherapy device used for tracking (e.g., the movement axes of the collimator and the movement axes of the patient positioning apparatus) are actuated accordingly in real time.

Adjusting the irradiation field to take account of the movement of the target volume solely using a collimator (e.g., by correctively adjusting the collimator aperture to track the movement of the target volume) may be unfavorable.

This is because with collimator-only tracking, as the collimator leaves have a certain width, the problem that a movement of the target volume occurring at right angles to the direction of the collimator leaves may not be compensated for precisely may arise, since the resolution predetermined by the leaf width precludes optimal beam correction. For that reason, a movement of the target volume occurring at right angles to the direction of the collimator leaves may not be compensated for precisely. This may lead to underdosages or overdosages in the surrounding healthy tissue.

The approach where the movement of the target volume during tracking is compensated for by a constant repositioning of the patient is also problematic. With this type of tracking, a patient table (e.g., with the patient positioned thereon) is displaced in order to keep the moving target volume effectively stationary in space. In this case, the planned isocenter of the target volume, seen from the treatment beam perspective, remains effectively at the same position or at the same position in the coordinate system of the radiotherapy device.

A disadvantage with the tracking using the patient table alone is that the inertia of the system is to be overcome. It is therefore difficult to adjust the position quickly. Adequate compensation of the movement of the target volume is therefore not always possible (e.g., in the case of large and rapid movement amplitudes, such as are observed with the respiratory movement). Changes affecting the shape of the target volume (e.g., deformations and/or rotations) may not be compensated for by a translation of the table alone.

This problem may be avoided with the aid of the disclosed radiotherapy device.

The tracking system that uses the collimator permits the treatment beam to be adjusted quickly to changes with respect to the position and/or the shape of the target volume. The collimator responds quickly to a movement of the target volume, and therefore, the irradiation field may quickly follow even large movements of the target volume through movement of the collimator leaves. The disadvantage of the collimator is that only a discrete resolution corresponding to the width of the collimator leaves (e.g., 5 mm or 1 cm) is possible at right angles to the movement direction of the leaves.

Movements in this direction, which lie between an integral multiple of the leaf width (e.g., a 5 mm leaf width: displacements by 2-3 mm or 7-8 mm), may only be tracked to an inadequate extent with the collimator; overdosages and underdosages would occur.

In contrast, tracking using the positioning apparatus (e.g., the patient table) permits accurate and precise movement of the patient without a predefined resolution as is preset in the case of the collimator.

Because tracking using the positioning apparatus is combined with tracking by the collimator, improved tracking may be provided. The collimator adjusts the treatment beam in a longitudinal direction, while the positioning apparatus adjusts the table in a transverse direction.

The disadvantages of collimator tracking in the transverse direction are accordingly canceled out. The compensation is still carried out solely in the transverse direction to a lesser degree such that the inertia problems that would occur in the case of tracking by the positioning apparatus alone are greatly reduced.

As a result of the combination of two different tracking systems (e.g., collimator tracking and patient table tracking), the different advantages and disadvantages of the two tracking systems are exploited, with the result that the advantages complement one another, and the disadvantages are neutralized.

The improved tracking method also permits narrower safety margins to be chosen, since the target volume may be irradiated with greater accuracy. In this way, the overall dose, to which the patient is exposed, may be reduced.

A further advantage of the tracking method is that a greater period of time for the irradiation is available than in the case of the gating method, in which only specific time windows in the movement cycle are available for irradiation purposes. For example, the irradiation may be carried out continuously.

An advantage compared to gating methods is produced in the case of irradiation techniques, in which the beam is emitted dynamically, as opposed to static “step-and-shoot” methods. With dynamic irradiation techniques, the gantry or the patient table, for example, is moved during the beam application.

These irradiation techniques may not be employed in a simple or obvious way with dynamic treatment methods. This is because in the case of dynamic treatment methods, the frequent and, within certain bounds, irregular interruption of the beam application is at odds with the continuous beam application employed in conjunction with simultaneous movement of dynamic components.

The tracking method avoids this disadvantage and therefore is well suited to being combined with dynamic irradiation techniques. For example, the subdivision of the corrective beam adjustment along two different movement directions over two different tracking systems permits accurate tracking to also be applied in the case of dynamic irradiation techniques.

The collimator is, for example, a multileaf collimator having a plurality of collimator leaves. The first movement direction may correspond to a movement direction of the collimator leaves.

The collimator therefore compensates for the change in the target volume in the direction of the collimator leaves of the collimator. This allows accurate tracking along the first movement direction.

The first movement direction and the second movement direction are arranged, for example, at right angles to each other. The movement of the target volume is therefore analyzed by the control apparatus to the effect that the movement of the target volume is subdivided along two movement directions at right angles to each other.

The first movement direction and the second movement direction may be aligned to the movement of the target volume such that the movement of the target volume along the first movement direction is stronger than the movement of the target volume along the second movement direction.

In this way, a large proportion of the movement of the target volume may be compensated for by the collimator, the leaves of which permit a rapid compensation in the movement direction of the leaves. The movement along the direction that is compensated for by the patient table is commensurately less pronounced.

The first movement direction may be aligned to the movement of the target volume such that the main movement direction of the target volume is aligned substantially parallel to the first movement direction. In this way, the corrective adjustment of the beam is realized primarily by the collimator or by a displacement of the collimator aperture. Thus, the direction of the collimator leaves may correspond to the main movement direction of the target volume. In this way, a large proportion of the movement of the target volume may be compensated for by the collimator, the leaves of which permit a rapid compensation in the movement direction of the leaves.

The positioning apparatus, in contrast, compensates for the movement of the target volume taking place substantially at right angles to the movement direction of the collimator leaves. Since this movement direction is not the main movement direction of the target volume, only more minor compensation movements of the positioning apparatus are provided. The minor compensation movements are to be handled by the positioning apparatus in spite of the inertia of the compensation.

For example, the second movement direction may be aligned such that only one movement axis of the positioning apparatus is activated in order to reposition the target volume along the second movement direction. This avoids a complex actuation of the patient couch that would require more than one movement axis to be moved simultaneously in a coordinated manner.

The method for correctively adjusting a beam for irradiating a moving target volume includes sensing a movement of the target volume, analyzing the movement of the target volume along a first movement direction and along a second movement direction, and correctively adjusting the beam in order to compensate for the movement of the target volume along the first movement direction by displacing a collimator aperture of a collimator along the first movement direction. The method also includes correctively adjusting the beam in order to compensate for the movement of the target volume along the second movement direction by repositioning the target volume along the second movement direction.

The collimator may be a multileaf collimator having a plurality of collimator leaves. The first movement direction corresponds to a movement direction of the collimator leaves. The first movement direction and the second movement direction are arranged, for example, at right angles to each other.

The collimator leaves are moved in parallel with the movement of the target volume in order to correctively adjust the treatment beam. In contrast, the patient positioning apparatus is moved in the opposite direction to the movement of the target volume in order to bring the target volume back into the focus of the treatment beam.

The first movement direction and the second movement direction are aligned to the movement of the target volume such that the target volume moves more strongly along the first movement direction than along the second movement direction.

The second movement direction is oriented such that only one movement axis of a positioning apparatus is activated in order to reposition the target volume along the second movement direction. In this case, the movement of the target volume is subdivided into the two movement directions such that one movement direction corresponds to a mechanical axis of the radiotherapy device.

For example, the first movement direction may be aligned at right angles to a movement axis of the positioning apparatus. The collimator is oriented at right angles to the movement axis.

For example, one movement axis may correspond to the longitudinal axis of the positioning apparatus, while the collimator is arranged perpendicularly with respect to the patient table. If the positioning apparatus is moved for tracking, only one movement axis of the positioning apparatus is to be moved. This is technically easier to realize than a diagonal, smooth movement with simultaneous use of two movement axes of the positioning apparatus.

The preceding and following description of the individual features, the advantages and the effects relates both to the device category and to the method category, without this being mentioned explicitly in each particular case. The individual features disclosed in the process may also be provided in other combinations than those shown.

### BRIEF DESCRIPTION OF THE DRAWINGS

FIG. 1 is a schematic diagram illustrating the problems with collimator-only tracking;

FIG. 2 is a diagram illustrating the problems with tracking only using a positioning apparatus;

FIG. 3 shows an exemplary division of the movement of the target volume along a main movement direction and a direction at right angles to the main movement direction;

FIG. 4 shows an exemplary division of the movement of the target volume into a first larger movement component and a second smaller movement component;

FIG. 5 shows the target volume in an exemplary first movement state with an associated collimator field;

FIG. 6 shows the target volume in an exemplary second movement state with the associated collimator field;

FIG. 7 shows the target volume in an exemplary second movement state, the target volume having been repositioned by a patient table, with the associated collimator field; and

FIG. 8 shows one embodiment of a radiotherapy device.

### DETAILED DESCRIPTION OF THE DRAWINGS

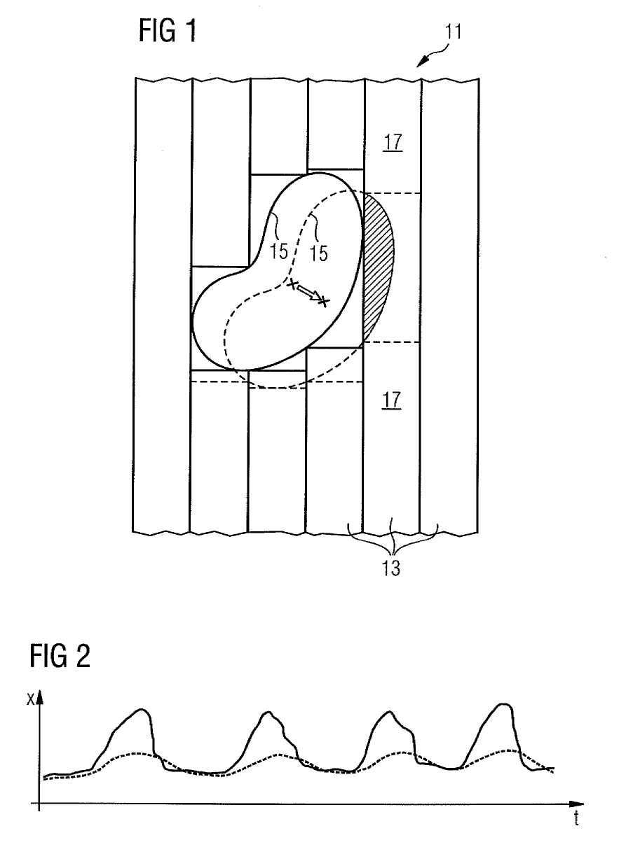

FIG. 1 shows a collimator 11 that suitably delimits the irradiation field for irradiating a target volume 15. A plurality of collimator leaves 13 that forms the irradiation field is shown.

The target volume 15 is shown drawn with a dashed outline at a second position. The movement executed by the target volume 15 between a first position and a second position has a component along a movement direction of the plurality of collimator leaves 13 and a movement direction at right angles to the movement direction of the plurality of collimator leaves 13.

If an irradiation field is formed for the displaced target volume 15, overdosages or underdosages may occur.

This may be illustrated by considering a pair of leaves 17. If the pair of leaves 17 is open, as indicated by the dashed lines, the tissue lying to the right of the target volume 15 is subjected to additional radiation. If the pair of leaves 17 remains closed, the hatched part of the target volume 15 is not irradiated.

FIG. 2 shows a diagram, in which the movement of the target volume in an x-direction is plotted against the time t. Given a correspondingly great movement amplitude of the target volume (continuous line) in the x-direction, the inertia of the positioning apparatus may result in the movement of the positioning apparatus (dotted line) lagging behind the movement of the target volume. A complex mechanism would be used in order to compensate for the inertia. The patient's comfort would be jeopardized if the movement is too rapid.

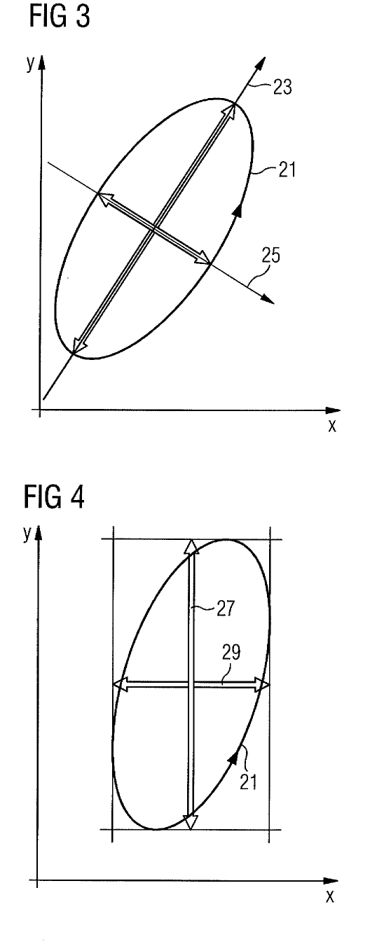

FIG. 3 shows a movement of the target volume in the x-y plane. The movement is symbolically indicated by an ellipse 21, the arrow along the ellipse 21 indicating the movement direction.

The movement of the target volume is strongest along a first direction 23 (e.g., a diagonal direction). This is the main movement direction of the target volume 15. The movement of the target volume 15 is at a minimum along the direction 25 at right angles to the first direction 23.

The collimator leaves are oriented in such a way that the leaves are aligned parallel to the first direction 23. The irradiation field is correctively adjusted along this direction solely by appropriate displacement of the collimator leaves.

With the positioning apparatus, in contrast, the target volume is repositioned along the second direction 25 in order to keep the target volume in the focus of the beam.

FIG. 4 shows the same movement of the target volume in the x-y plane. The movement is subdivided differently (e.g., along the coordinate system). Although the movement of the target volume is not strongest along the y-direction, the movement of the target volume along the y-direction is stronger than in the x-direction.

The collimator leaves are oriented such that the leaves are aligned parallel to the y-direction. The irradiation field is correctively adjusted along this first direction 27 by appropriate displacement of the collimator leaves.

With the positioning apparatus, in contrast, the target volume is repositioned along the direction 29 at right angles to the first direction 27 (e.g., the x-direction) in order to keep the target volume in the focus of the beam.

An implementation such as this may have the advantage over the implementation shown in FIG. 3 that it is easier to realize, since the collimator and/or the positioning apparatus do not have to adjust exactly according to the strongest movement direction of the target volume. However, tracking according to the implementation shown in FIG. 3 exploits the advantages of the combined tracking system in terms of movability of the collimator leaves and inertia of the positioning apparatus to best effect.

FIG. 5 shows the target volume 15 in a first movement state, and the associated collimator field generated by the collimator 11 from the perspective of the treatment beam. The positioning apparatus 31 (e.g., a patient table, on which the target volume 15 is positioned for the irradiation) is indicated underneath the collimator field.

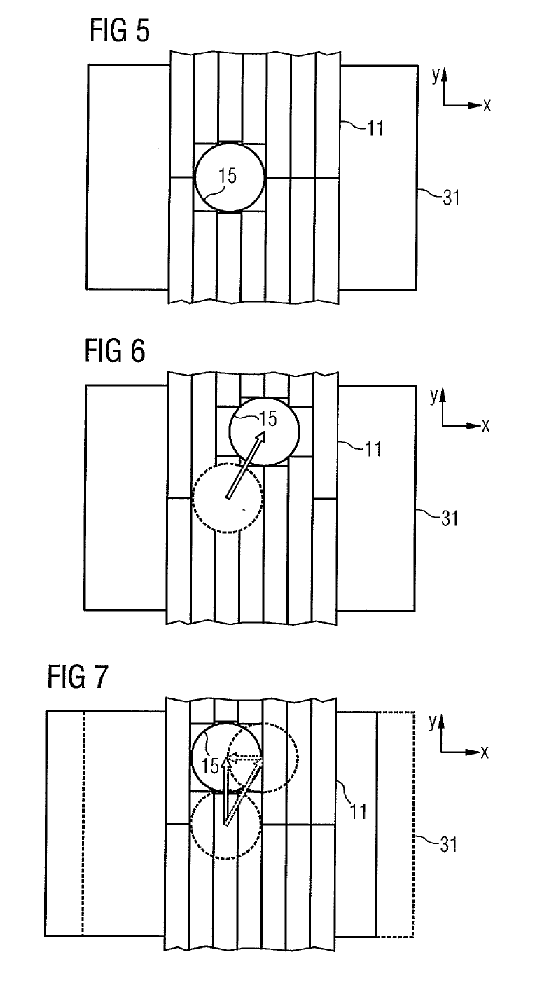

FIG. 6 shows the target volume 15 in a second movement state with the associated collimator field. The target volume has shifted on the positioning apparatus 31 such that other collimator leaves would be used in order to suitably delimit the irradiation field.

FIG. 7 shows the target volume 15 in the second movement state. The target volume 15 has been repositioned back by the positioning apparatus 31 and along a movement direction standing at right angles to the displacement direction of the collimator leaves (e.g., the x-direction). The arrows show the breakdown of the movement into the two components, each of which is compensated for by one of the tracking subsystems.

Only a displacement of the collimator leaves already used in FIG. 5 is provided in order to suitably delimit the irradiation field. If there is an additional change in shape of the target volume, more or fewer collimator leaves may be provided in certain cases in order to suitably delimit the irradiation field.

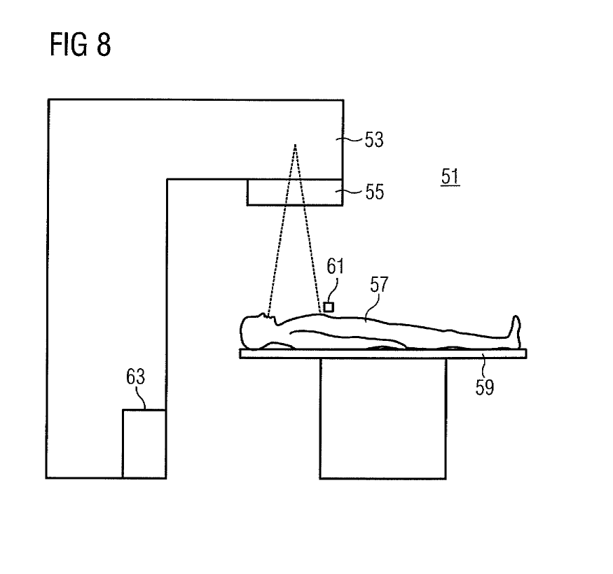

FIG. 8 shows a radiotherapy device 51, with which the corrective beam adjustment may be carried out using the two tracking subsystems, as described.

The therapeutic radiation source is located, for example, in an L-shaped gantry 53. A collimator 55 is mounted on the gantry 53.

The patient 57 (or a phantom for testing the corrective beam adjustment) is located on a patient couch 59. A position of the patient 57 or the phantom may be adjusted with different degrees of freedom.

A motion sensor 61 supplies a signal, on the basis of which the movement of the target volume to be irradiated may be determined.

A control apparatus 63 that has an input for acquiring the signal breaks down the movement of the target volume into two components that are to be compensated using different tracking subsystems. In this arrangement, one tracking subsystem is realized by the correspondingly actuated collimator 55, and the other tracking subsystem is realized by the correspondingly actuated patient couch 59.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A radiotherapy device comprising
a therapeutic radiation source operable to provide a beam for irradiating a target volume that is moving; and
a beam correcting apparatus operable to direct the beam onto the target volume,
wherein the beam correcting apparatus comprises:
a collimator comprising a collimator aperture operable to delimit the beam;
a positioning apparatus operable to position the target volume relative to the beam; and
a controller configured to:
select a position of the collimator aperture relative to the beam; and
select a position of the positioning apparatus such that when the beam is correctively adjusted, movement of the target volume in a first movement direction is compensated for by displacing the collimator aperture, and movement of the target volume in a second movement direction is compensated for by repositioning the target volume with the positioning apparatus.

2. The radiotherapy device as claimed in claim 1, wherein the collimator is a multileaf collimator comprising a plurality of collimator leaves, and
wherein the first movement direction corresponds to a movement direction of the plurality of collimator leaves.

3. The radiotherapy device as claimed in claim 1, wherein the first movement direction and the second movement direction stand at right angles to each other.

4. The radiotherapy device as claimed in claim 1, wherein the first movement direction and the second movement direction are aligned to the movement of the target volume such that the movement of the target volume along the first movement direction is stronger than the movement of the target volume along the second movement direction.

5. The radiotherapy device as claimed in claim 4, wherein the first movement direction is aligned to the movement of the target volume such that a main movement direction of the target volume is aligned substantially parallel to the first movement direction.

6. The radiotherapy device as claimed in claim 1, wherein the second movement direction is aligned such that only one movement axis of the positioning apparatus is activated in order to reposition the target volume along the second movement direction.

7. The radiotherapy device as claimed in claim 2, wherein the first movement direction and the second movement direction stand at right angles to each other.

8. The radiotherapy device as claimed in claim 2, wherein the first movement direction and the second movement direction are aligned to the movement of the target volume such that the movement of the target volume along the first movement direction is stronger than the movement of the target volume along the second movement direction.

9. The radiotherapy device as claimed in claim 8, wherein the first movement direction is aligned to the movement of the target volume such that a main movement direction of the target volume is aligned substantially parallel to the first movement direction.

10. The radiotherapy device as claimed in claim 5, wherein the second movement direction is aligned such that only one movement axis of the positioning apparatus is activated in order to reposition the target volume along the second movement direction.

11. A method for correctively adjusting a beam for irradiating a moving target volume, the method comprising:
sensing a movement of the target volume;
analyzing the movement of the target volume along a first movement direction and along a second movement direction;
correctively adjusting the beam in order to compensate for the movement of the target volume along the first movement direction by displacing a collimator aperture of a collimator along the first movement direction; and
correctively adjusting the beam in order to compensate for the movement of the target volume along the second movement direction by repositioning the target volume along the second movement direction.

12. The method as claimed in claim 11, wherein the collimator is a multileaf collimator having a plurality of collimator leaves, and
wherein the first movement direction corresponds to a movement direction of the collimator leaves.

13. The method as claimed in claim 11, wherein the first movement direction and the second movement direction stand at right angles to each other.

14. The method as claimed in claim 11, wherein the first movement direction and the second movement direction are aligned to the movement of the target volume such that the target volume moves more strongly along the first movement direction than along the second movement direction.

15. The method as claimed in claim 14, wherein the first movement direction is aligned substantially parallel to a main movement direction of the target volume.

16. The method as claimed in claim 11, wherein the second movement direction is aligned such that only one movement axis of a positioning apparatus is activated in order to reposition the target volume along the second movement direction.

17. The method as claimed in claim 12, wherein the first movement direction and the second movement direction stand at right angles to each other.

18. The method as claimed in claim 12, wherein the first movement direction and the second movement direction are aligned to the movement of the target volume such that the target volume moves more strongly along the first movement direction than along the second movement direction.

19. The method as claimed in claim 18, wherein the first movement direction is aligned substantially parallel to a main movement direction of the target volume.

20. The method as claimed in claim 12, wherein the second movement direction is aligned such that only one movement axis of a positioning apparatus is activated in order to reposition the target volume along the second movement direction.
